(19) United States
(12) Patent Application Publication
(10) Pub. No.: US 2009/0125248 A1
(43) Pub. Date: May 14, 2009

(54) System, Method and computer program product for integrated analysis and visualization of genomic data

(76) Inventors: Soheil Shams, Manhattan Beach, CA (US); James Darrell Park, Vail, AZ (US); Viren Wasnikar, Los Angeles, CA (US); Razmik Shahinian, Los Angeles, CA (US)

Correspondence Address: TOPE-MCKAY & ASSOCIATES, 23852 PACIFIC COAST HIGHWAY #311, MALIBU, CA, 90265, US

(21) Appl. No.: 12/291,523

(22) Filed: Nov. 10, 2008

Related U.S. Application Data
(60) Provisional application No. 61/002,418, filed on Nov. 9, 2007.
(60) Provisional application No. 61/003,722, filed on Nov. 20, 2007.

(51) Int. Cl. G06F 19/00; G01N 33/48

(52) U.S. Cl. 702/20

(57) ABSTRACT

Described is a system for analysis and visualization of genomic data. The system allows a user to select at least one individual sample. The sample has chromosomal data representing a genome with a chromosome and also includes chromosomal measurements of at least one event at a particular location on the chromosome. A frequency of event is generated based on the selected sample. The frequency of event is a frequency of occurrence of the event in the selected sample. At least one annotation can be selected that includes chromosomal region specific information as related to the chromosome. Finally, the chromosomal data, the annotation, and the frequency of event on a display can all be simultaneously displayed, thereby allowing a user to view chromosomal region specific information with respect to a particular chromosomal event.

### PRIORITY CLAIM

The present application is a non-provisional patent application, claiming the benefit of priority of U.S. Provisional Application No. 61/002,418, filed on Nov. 9, 2007, entitled, “Integrated Visualization and Analysis Tool for Genomic Data,” and U.S. Provisional Application No. 61/003,722, filed on Nov. 20, 2007, entitled, “System and method for application of gene set enrichment analysis to DNA copy number data.”

### FIELD OF INVENTION

The present invention relates to an analysis and visualization system and, more particularly, to a system for the integrated analysis and visualization of genomic data.

### BACKGROUND OF INVENTION

Genomic visualization tools have been devised to assist researchers, laboratories, and other users to visually display and understand genomic data. The genomic data is often in the form of individual samples having chromosomal data (including measurements of at least one event at a particular location on the chromosomes). An event here would indicate some measurement related to the genome. Examples of such measurements include the expression of a gene, an exon at a particular location, the number of copies of a portion of the genome that have been gained or lost, the extent of methylation of the genome at a particular location, the affinity of certain promoters to bind to a particular area on the genome, etc. In some cases, users may calculate a frequency of event based on a frequency of occurrence of the event in the selected sample. For example, it may be desirable to calculate the frequency of aberration, such as the frequency of a gain or loss of chromosomal copies when compared to a reference sample in a selected population of samples. In other circumstances, it may be desirable to review an annotation regarding specific information as related to a particular chromosomal region of the chromosome. Such information might include items such as what genes are present in a location and if there are known copy number polymorphisms in that area (including a list of such polymorphisms). Other items might include information pertaining to the presence of miroRNAs and potential Single Nucleotide Polymorphism (SNP)s in the area, etc.

The existing systems available for visualization of chromosomal or genomic annotations, such as the University of California of Santa Cruz (U.C.S.C.) browser (reference) and the Ensemble Genome Browser (reference), display various annotations for a specific region of the genome. Ensemble is a joint project between the European Molecular Biology Laboratory (EMBL), the European Bioinformatics Institute (EBI) and the Wellcome Trust Sanger Institute (WTSI).

Alternatively, a user may calculate a frequency of event and thereafter display the frequency on a separate screen. While functional, existing visualization tools do not readily integrate such genomic annotations with user supplied sample data indicating chromosomal events per sample. Further and of notable importance, existing tools do not allow for a seamless integration between the frequency of events for the user selected set of samples along with the samples and genomic annotation data.

Thus, a continuing need exists for a system that simultaneously displays and integrates genomic data pertaining to individual samples, a frequency of event, and annotations. A need further exists for additional integrated features, such as sorting the samples, displaying the sample annotations, creating factor aggregate plots of the samples, etc. The present invention solves these needs as described below.

### SUMMARY OF INVENTION

The present invention relates to a system, method, and computer program product for the integrated analysis and visualization of genomic data. The method includes several acts, including selecting at least one individual sample, the sample having chromosomal data representing a genome with a chromosome and including chromosomal measurements of at least one event at a particular location on the chromosome. A frequency of event is generated based on the selected sample. The frequency of event is a frequency of occurrence of the event in the selected sample. At least one annotation is selected. The annotation includes chromosomal region specific information as related to the chromosome. Finally, the chromosomal data, the annotation, and the frequency of event are displayed on a display, thereby allowing a user to view chromosomal region specific information with respect to a particular chromosomal event.

In another aspect, the event is a gain or loss of chromosomal copies in the selected sample as compared against a reference chromosomal sample, such that the chromosomal measurements represent chromosomal copies that are gained or lost.

The present invention also includes an act of zooming into a selected region of the genome to illustrate chromosomal measurements in the selected region, a corresponding frequency of event in the selected region, and corresponding chromosomal region specific information.

Additionally, the gains and losses of chromosomal copies are displayed as bars having heights that extend from a median line. The median line represents the reference chromosomal sample and the height of the bars represents copies that are gained or lost from the reference chromosomal sample.

The present invention also includes an act of selecting a plurality of samples such that the frequency of event is based on the selected samples, with the frequency of event being a frequency of occurrence of the event across the selected samples.

In yet another aspect, the present invention includes an act of selecting a particular chromosomal event and location from the display of the frequency of event. The chromosomal event at the selected location spans a region of the chromosome, where the spanned region has a span length. Additionally, the samples are sorted according to each sample's span length with respect to the selected event.

Additionally, in the act of selecting a plurality of samples, each sample is labeled with at least one factor having a factor value. Additional acts include selecting a factor with respect to the selected samples; grouping the selected samples such that the selected samples having the same factor values are grouped together; and generating and displaying a frequency of event for each group of samples.

In yet another aspect, the event is an chromosomal event selected from a group consisting of an allele gain or loss in the selected sample as compared against a reference chromosomal sample, gene expression and determining if the gene is up regulated or down regulated, a methylated event and determining if the gene is hyper or hypo methylated, and a binding event and determining if there exists a promoter binding or promoter unbinding.

In another aspect, the present invention includes a method for measuring similarity between samples based on genomic data. The method includes acts of electing a plurality of individual samples, where each sample includes chromosomal data representing a genome with a chromosome and including chromosomal measurements of at least one event at a particular location on the chromosome. A frequency of event is generated for each sample, the frequency of event being a frequency of occurrence of the event in the selected sample. An aggregate profile is generated of the genome, the aggregate profile formed of a plurality of samples and representing a percentage of samples having a particular event at each location along the genome. The genome is subdivided into intervals, where each interval has a constant frequency of event. A weighting function is assigned to each interval. A feature vector is set equal to the weighting function for each sample at each event location. A distance measure is calculated between a pair of samples based on the feature vectors of each sample. A distance matrix is generated showing a distance between any pair of samples. Finally, the samples are clustered based on the distance matrix such that samples with distances below a predetermined threshold are clustered together.

In another aspect, the present invention includes a method for integrated analysis of copy number and expression data. The method comprises acts of:
selecting a genome of interest, the genome of interest having a total of N genes;
selecting a region R with a copy number change greater than a predetermined threshold, the region R having a total of X genes that fall completely within region R or partly cover region R;
identifying Y genes that are to be differentially regulated within region R; and
determining if the Y genes that are to be differentially regulated are differentially regulated at a rate greater than pure chance according to the following:
   wherein the probability of drawing X genes at random from the original population and ending up with exactly Y differentially expressed genes is:

$$\frac{( \begin{matrix}M \\ Y\end{matrix} ) ( \begin{matrix}N - M \\ X - Y\end{matrix} )}{( \begin{matrix}N \\ X\end{matrix} )}$$

such that the probability (p-value) of getting at least Y differentially expressed genes is:

$${\sum }_{j = Y}^{X} \frac{( \begin{matrix}M \\ j\end{matrix} ) ( \begin{matrix}N - M \\ X - j\end{matrix} )}{( \begin{matrix}N \\ X\end{matrix} )} ; and$$

calculating a false discover rate corrected Q-value using the p-value.

Finally, the present invention also includes a computer program product and system. The computer program product comprises computer-readable instruction means stored on a computer-readable medium that are executable by a computer having a processor for causing the processor to perform the operations describe herein. The system includes one or more processors that are configured to perform the operations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The objects, features and advantages of the present invention will be apparent from the following detailed descriptions of the various aspects of the invention in conjunction with reference to the following drawings, where:

FIG. 1 is a block diagram depicting the components of a system for integrated analysis and visualization of genomic data according to the present invention;

FIG. 2 is an illustration of a computer program product according to the present invention;

FIG. 3 is an illustration of a screenshot of a visualization tool according to the present invention, illustrating a genome-level view of individual samples, annotations, and a frequency of event;

FIG. 4 is an illustration of a screenshot of a visualization tool according to the present invention, illustrating detailed information as related to a particular selected sample;

FIG. 5 is an illustration of a screenshot of a visualization tool according to the present invention, illustrating detailed information as related to a particular selected chromosome;

FIG. 6 is an illustration of a screenshot of a visualization tool according to the present invention, illustrating a summary of detailed information as related to a selected sample;

FIG. 7 is an illustration of a screenshot of a visualization tool according to the present invention, illustrating detailed information as related to a whole genome;

FIG. 8 is an illustration of a screenshot of a visualization tool according to the present invention, illustrating a chromosome-level view of individual samples, annotations, and a frequency of event;

FIG. 9 is an illustration of a screenshot of a visualization tool according to the present invention, illustrating a chromosome-level view with the individual samples sorted according to a frequency of event;

FIG. 10 is an illustration of a screenshot of a visualization tool according to the present invention, illustrating a sample selection screen where a user can select samples to view with the visualization tool;

FIG. 11 is an illustration of a screenshot of a visualization tool according to the present invention, illustrating that each sample is labeled with at least one factor having a factor value and that the samples can be selected and grouped according to the factor values;

FIG. 12 is an illustration of a screenshot of a visualization tool according to the present invention, illustrating a particular factor value;

FIG. 13 is an illustration of a screenshot of a visualization tool according to the present invention, illustrating sample aggregates, where all samples having a common factor value are grouped together and displayed as a frequency plot;

FIG. 14 is an illustration of a screenshot of a visualization tool according to the present invention, illustrating differentially regulated genes;

Appendix A is a paper by the inventors of the present invention, entitled, “Copy Number Computation;”

Appendix B is a paper by the inventors of the present invention, entitled, “Integrated Analysis of Copy Number and Expression Data;”

Appendix C is a paper by the inventors of the present invention, entitled, “Application of Gene Set Enrichment Analysis to DNA Copy Number Data;”

Appendix D is a paper by the inventors of the present invention, entitled, “Clustering Genomic Profiles;”

Appendix E is a paper by the inventors of the present invention, entitled, “SNPRank: Segmentation from SNP Data;” and

Appendix F is a user's manual of a system incorporating the present invention, including descriptions of features and functions of the present invention.

### DETAILED DESCRIPTION

The present invention relates to an analysis and visualization system, and more particularly, to a system for the integrated analysis and visualization of genomic data. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

Before describing the invention in detail, first a description of various principal aspects of the present invention is provided. Subsequently, specific details of the present invention are provided to give an understanding of the specific aspects.

(1) Principal Aspects

The present invention has three “principal” aspects. The first is system for analysis and visualization of genomic data. The system is typically in the form of a computer system (with one or more processors) operating software or in the form of a “hard-coded” instruction set. This system may be incorporated into a wide variety of devices that provide different functionalities. The second principal aspect is a method, typically in the form of software, operated using a data processing system (computer). The third principal aspect is a computer program product. The computer program product generally represents computer-readable instruction means stored on a computer-readable medium such as an optical storage device, e.g., a compact disc (CD) or digital versatile disc (DVD), or a magnetic storage device such as a floppy disk or magnetic tape. Other, non-limiting examples of computer-readable media include hard disks, read-only memory (ROM), and flash-type memories. These aspects will be described in more detail below.

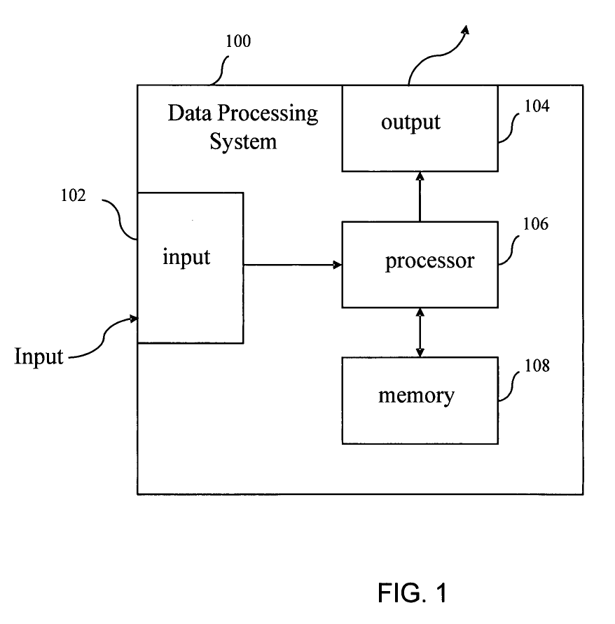

A block diagram depicting the components of system for analysis and visualization of genomic data according to the present invention is provided in FIG. 1. The system 100 comprises an input 102 for receiving information from a user or information regarding the data samples. Note that the input 102 may include multiple “ports.” An output 104 is connected with the processor for providing information regarding the genomic data to a user (e.g., through a display) or to other systems in order that a network of computer systems may serve as an analysis and integration system. Output may also be provided to other devices or other programs; e.g., to other software modules, for use therein. The input 102 and the output 104 are both coupled with a processor 106, which may be a general-purpose computer processor or a specialized processor designed specifically for use with the present invention. The processor 106 is coupled with a memory 108 to permit storage of data and software that are to be manipulated by commands to the processor 106.

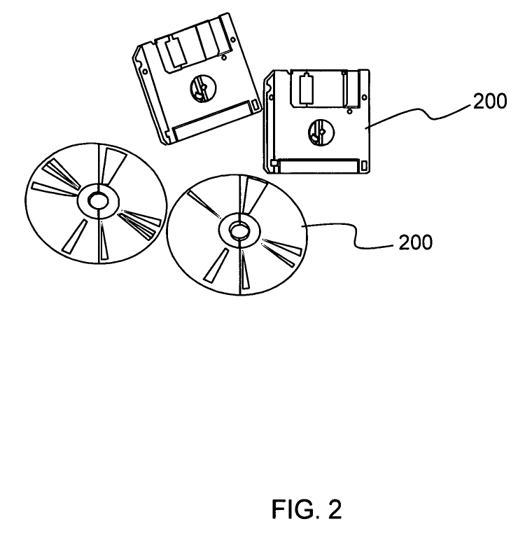

An illustrative diagram of a computer program product embodying the present invention is depicted in FIG. 2. The computer program product 200 is depicted as an optical disk such as a CD or DVD. However, as mentioned previously, the computer program product generally represents computer-readable instruction means stored on any compatible computer-readable medium. The term “instruction means” as used with respect to this invention generally indicates a set of operations to be performed on a computer, and may represent pieces of a whole program or individual, separable, software modules. Non-limiting examples of “instruction means” include computer program code (source or object code) and “hard-coded” electronics (i.e., computer operations coded into a computer chip). The “instruction means” may be stored in the memory of a computer or on a computer-readable medium such as a floppy disk, a CD-ROM, and a flash drive.

(2) Specific Details

The present invention is related to a system for the integrated analysis and visualization of genomic data. The system is generally configured to receive data and allow a user to manipulate the data for easy visualization and analysis upon a display (e.g., computer screen). The system also allows for the integration of the data by allowing the manipulation of one type of data to be reflected across the varying forms of genomic data.

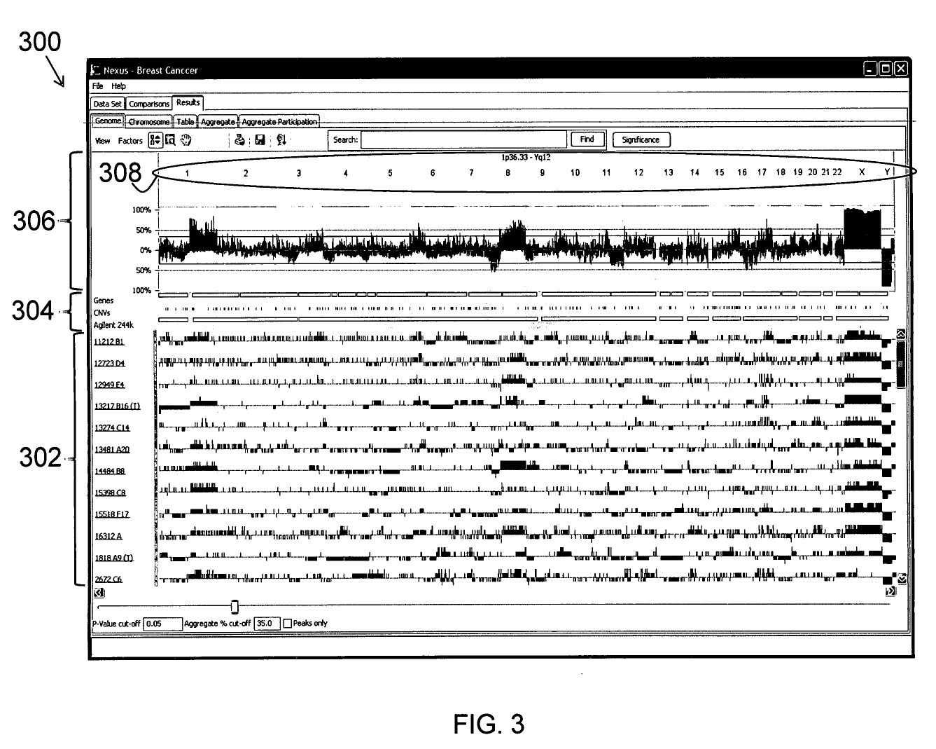

For example, FIG. 3 illustrates a screen shot of a user interface 300 for viewing and manipulating various genomic data. FIG. 3 illustrates a genome-level view of individual samples 302, annotations 304, and a frequency of event 306. The bottom part of the display shows each individual sample 302, one per row. As can be appreciated by one skilled in the art, while the samples 302 are illustrated at the bottom and the frequency of event 306 is illustrated at the top of the display, the present invention is not intended to be limited thereto as the various items can be moved around the display per the user's (or designer's) particular needs.

In a “whole genome” view as illustrated in FIG. 3, all the chromosomes 308 are shown at once, with the chromosomes laid horizontally and one after the other. Each selected sample 302 includes chromosomal data representing a genome with a chromosome 308 and includes chromosomal measurements of at least one event at a particular location on the chromosome 308. The chromosomal events are any chromosomal level events that are measurable. For example, the chromosomal events can be chromosomal gains and losses as compared to a reference sample. Other non-limiting examples of chromosomal events include allele gain or loss in the selected sample as compared with a reference chromosomal sample, gene expression and whether or not the gene is up regulated or down regulated, a methylation event and whether or not the gene is hyper- or hypo-methylated compared to a reference sample, and a binding event indicating whether or not there exists a particular promoter binding at particular chromosomal location.

The chromosomal measurements of the chromosomal events can be illustrated along each sample 302. As a non-limiting example, for each sample 302, a green segment above the median line indicates a chromosomal gain and a red bar under the median shows a chromosomal loss (as compared to a reference sample). The height of the bar is related to the number of copies gained or lost (e.g., higher bars show higher number of copies). It should be understood that any colors or orientations described herein are not intended to be limiting but are used for illustrative purposes and can be interchanged with outer suitable colors and/or orientations.

On the same display screen and above (or below, etc.) the samples 302 are the genome annotation 304 “tracks”. Here, various annotations 304 of the genome can be plotted. The annotations 304 include chromosomal region specific information as related to the chromosome and samples 302. As a non-limiting example, gene names can be displayed in a first track while a second track is used to show the areas of known copy number variations (marked by magenta colored bars). Finally, a third track can be used to illustrate tick marks for the location of array probes along the genome. Additional tracks can be added or removed by the user.

The top area of the screen 300 is used to display the frequency of event 306. The frequency of event 306 is based on the selected sample(s) and is the frequency of occurrence of the event in the selected samples. As a specific example, each point along the genome has a frequency of aberration based on the selected sample. As a non-limiting example, if a particular point along the genome is deleted in 30% of the samples, then the frequency of event 306 at that point would be 30% and shown as a red bar below the median line.

As noted above, the present invention is fully integrated to allow for easy analysis. For example, the samples 302 are drawn as hyperlinks so that when the user clicks on an individual sample, the user interface provides more detailed information about the selected sample.

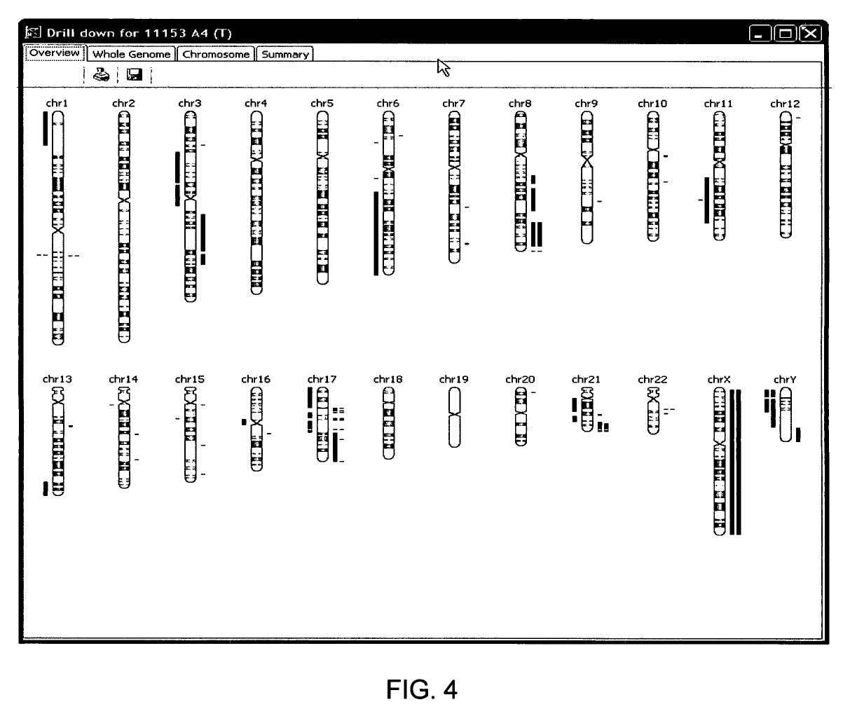

For example, FIG. 4 is an illustration of a screenshot depicting detailed information as related to a particular selected sample. FIG. 4 illustrates chromosomal events for the selected sample, along with associated ideograms.

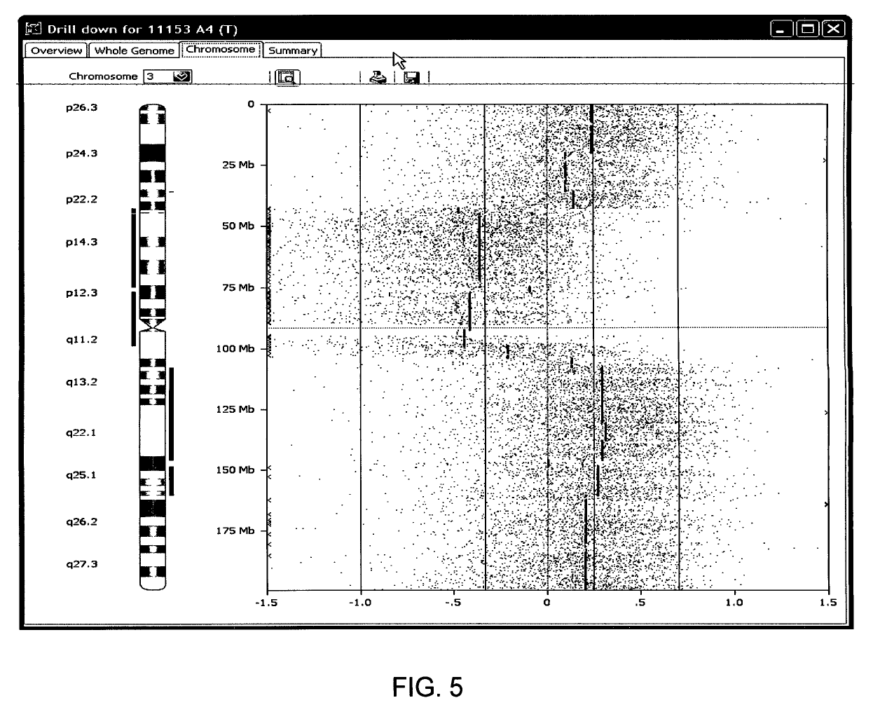

FIG. 5 is an illustration of a screenshot, depicting detailed information as related to a particular selected chromosome, including probe-level data, close-up views of the segmentation results, parameters, genomic locations and ideograms for the selected chromosome.

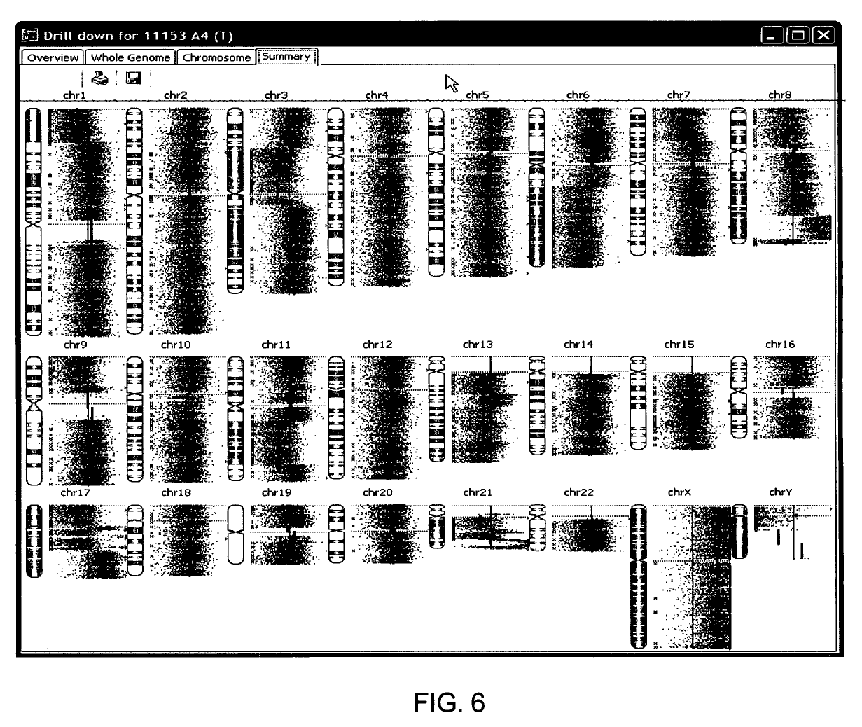

FIG. 6 is an illustration of a screenshot, depicting a summary of the detailed information as related to the selected sample, including probe-level data and chromosomal events shown as colors on the ideograms for the entire genome.

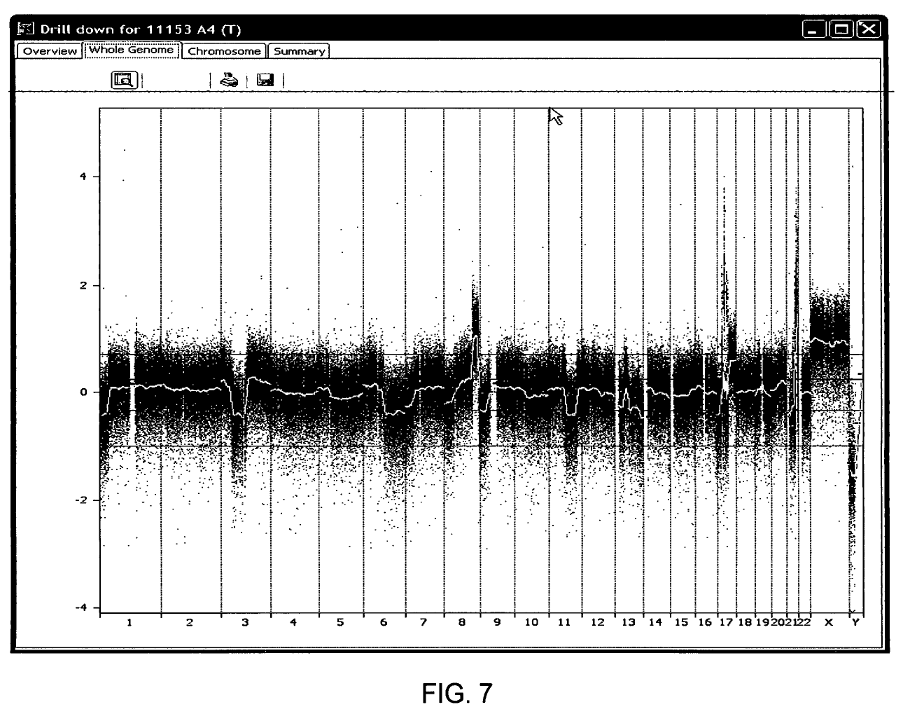

FIG. 7 is an illustration of a screenshot, depicting a whole genome view of the data for the selected samples. FIG. 7 illustrates probe-level data for the entire genome along with segmentation results, the moving average of probe log-ratio values, and cut-offs used for making calls on events.

Throughout the various displays, the computer pointer (and pointer device (e.g., mouse)) is used to display various pieces of information when moved around the display. For example, if on the frequency plot area (i.e., frequency of event 306), the tool-tip will indicate the actual frequency of the event (gain if above the median and loss if below (or vice versa)) at that location. When the tool tip is on the sample area 302, it shows the genomic position and sample name.

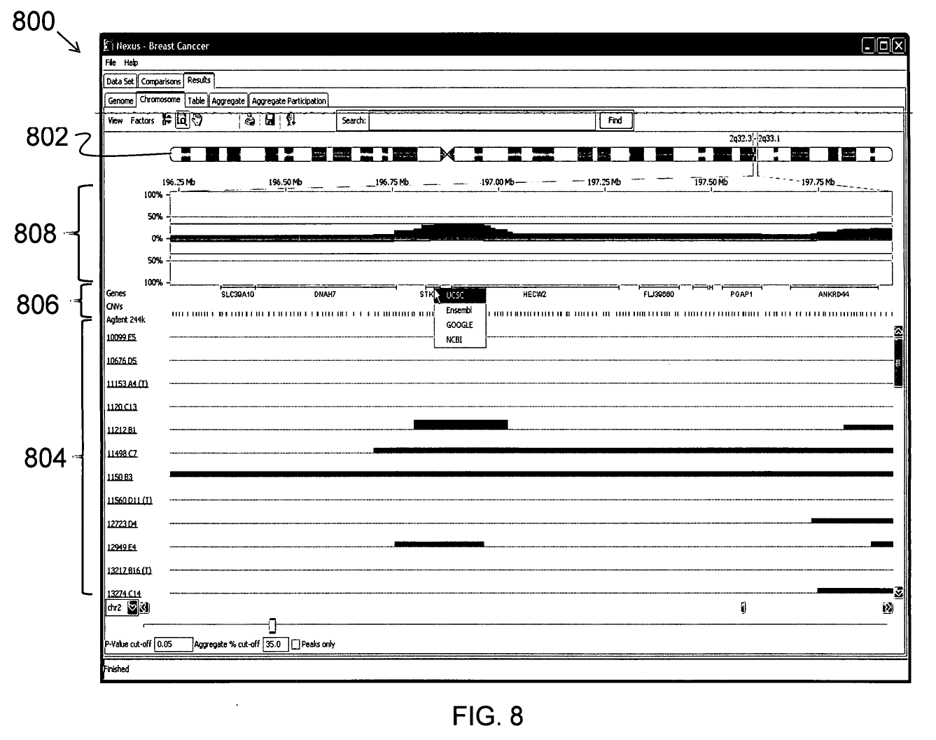

A display similar to that of FIG. 3 is used to illustrate the same information per selected chromosome, as shown in FIG. 8. FIG. 8 illustrates a screen shot 800 with information pertinent to a selected chromosome 802. Also illustrated are the selected samples 804 (depicting the selected chromosome information for each selected sample), annotations 806, and a corresponding frequency of event 808. Also as depicted, a user can use a zoom tool to zoom into any area on the genome and once sufficiently zoomed in, can see the gene names or any other selected annotation 806. It should be noted that this function and all functions for the chromosome are also available for the whole genome tab, as shown in FIG. 3. The user can then select one of the public databases to search for further information by using the mouse and clicking on the gene name.

It should be noted that when zooming, the illustrated samples 804 and corresponding frequency of event 808 are both zoomed to maintain a scale between the two illustrations as well as displaying the genomic annotations covering the range of the genome being viewed.

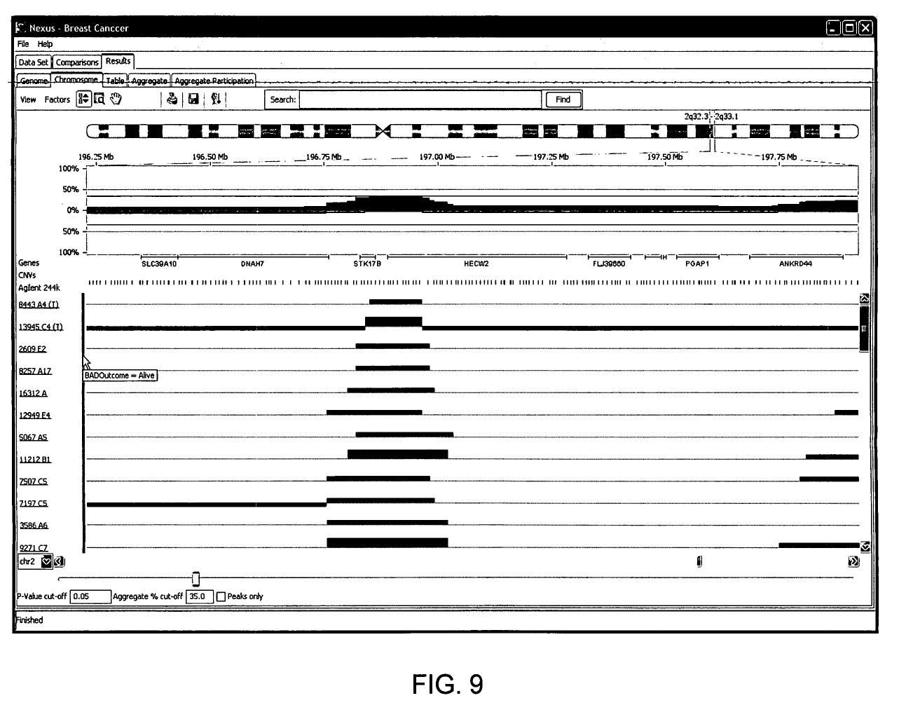

In another aspect, the present invention allows a user to sort the samples with a sort tool. For example and as illustrated in FIG. 9, when the user clicks on a particular point on the genome with an event (e.g., gain or loss), all samples having that event are sorted such that the sample with the smallest such aberration is sorted to the top and the longer/larger ones are sorted farther down. Thus, a user can select a particular chromosomal event and location from the display of the frequency of event and quickly identify samples that exhibit the selected event at the particular genomic position selected by the user. As can be appreciated by one skilled in the art, the chromosomal event at the selected location spans a region of the chromosome and the spanned region has a span length. Therefore, when sorting, the samples can be sorted according to each sample's span length with respect to the selected event. As a specific non-limiting example, the samples can be sorted by genomic aberration. In this aspect, the bottom of the sort are those samples that have an event in the opposite direction. For example, instead of a gain, the samples have a loss. It should be understood that the samples can be sorted using a variety of sampling criteria that are reflective of a selected event.

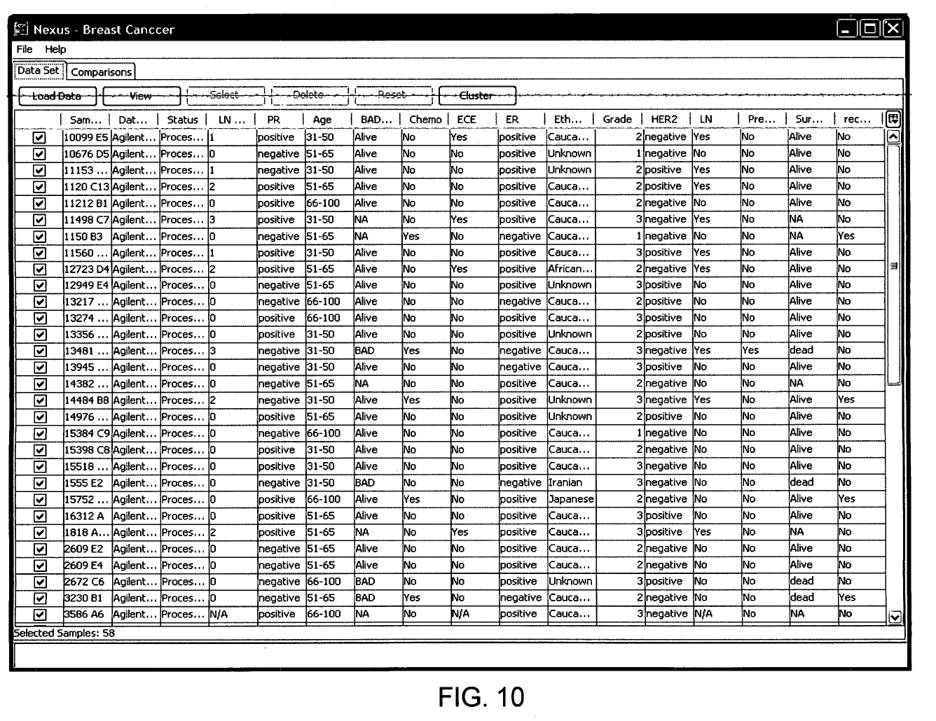

FIG. 10 illustrates a dataset tab consisting of a table showing various samples and their respective attributes or factors. This table allows a user to choose which samples to display and analyze by selecting them in the dataset tab. As a non-limiting example, the dataset tab will illustrate all available samples. Upon selecting some (or all) of the samples, the selected samples are then illustrated alongside the annotations and frequency of event (as shown in FIG. 3). Additionally, when selecting samples, it may be beneficial to first sort the samples. Thus, the present invention is configured to sort the samples in the dataset based on any factor (e.g., clinical parameters such as tumor grade, etc.). Such sorting will be reflected in the order in which samples are displayed in FIG. 3 (i.e., area 302). The user can select the samples to visualize and process by using the check box selection (or any other suitable selection technique).

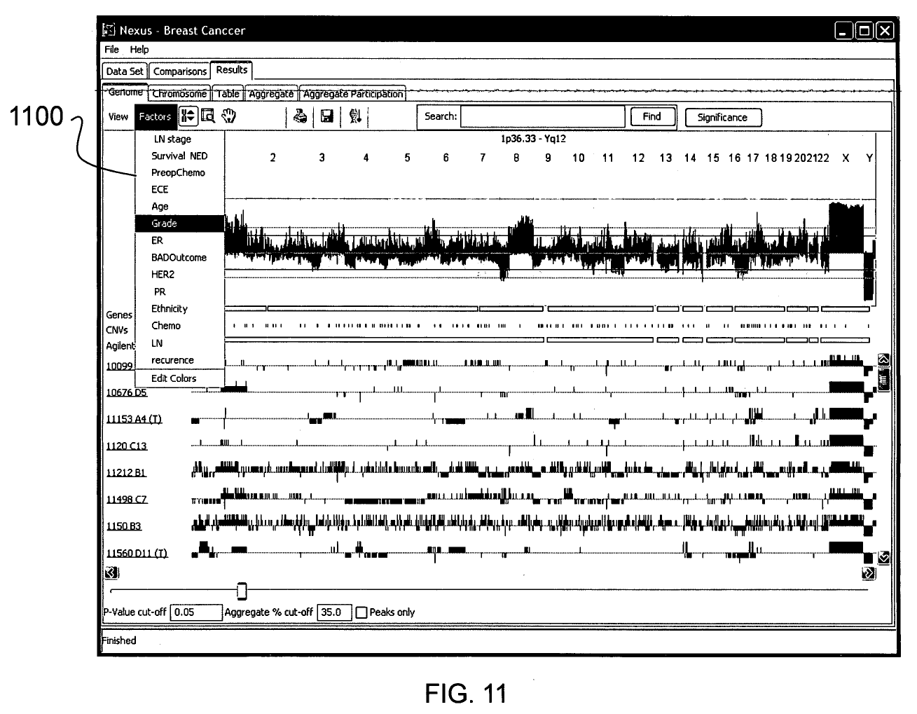

In another aspect, the system is configured to allow a user to visualize the factor values associated with each sample (in the whole genome view (e.g., FIG. 3) and chromosome view (e.g., FIG. 8)) by selecting the factor from a factor menu. The factor is any suitable variable or label that can be associated with a particular sample, non-limiting examples of which include age, sex, ethnicity, recurrence, chemotherapy treated, etc. As shown in FIG. 11, a factor menu 1100 is provided to allow a user to select a factor with respect to the selected samples.

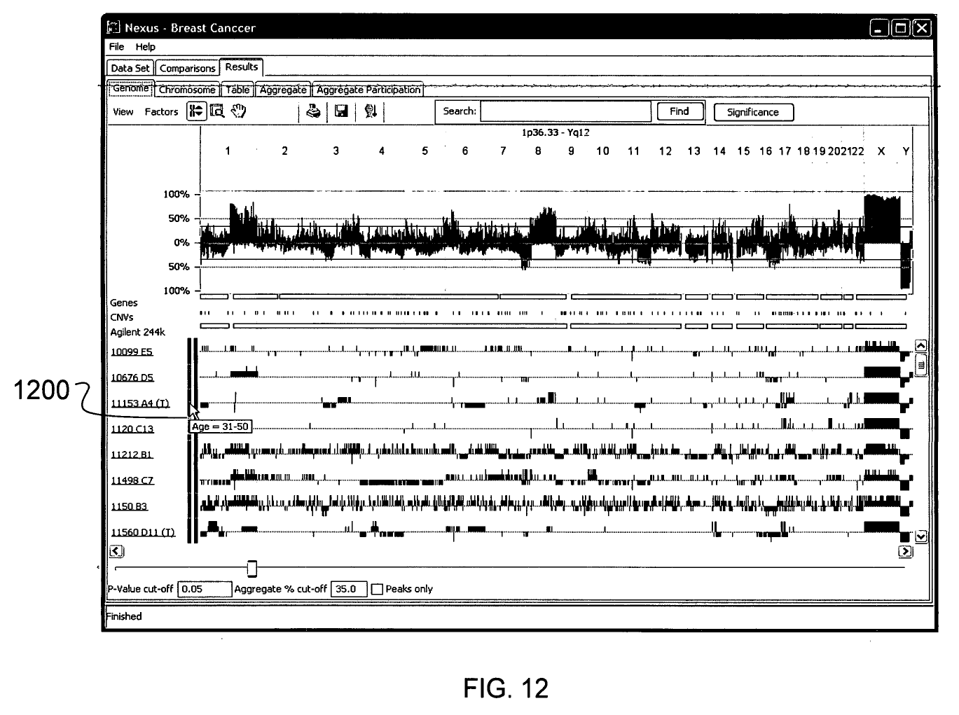

Additionally, the system is configured to show the factor value corresponding to the selected factor for each sample in the display area 302. Furthermore, the system is configured to allow a user to select multiple factors at the same time. For example, the factor menu listed above can be used to select multiple factors, which are displayed using any suitable technique. As a non-limiting example and as shown in FIG. 12, the multiple factors can be illustrated using colored lines 1200 that are next to the samples. Moving the mouse over the colored lines 1200 will provide the corresponding factor value.

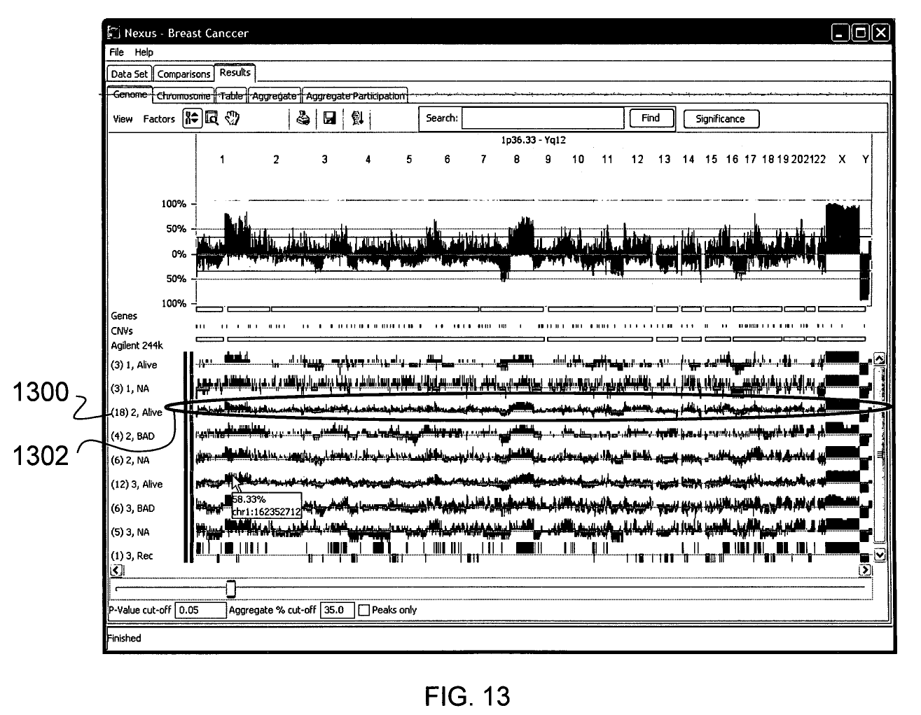

In another aspect and as shown in FIG. 13, the samples that are depicted in the bottom section of the display can be changed from showing individual samples to displaying “Sample Aggregates” 1300. A “View” menu is provided to select between the individual and sample aggregate views. Here all the samples having the same factor values are grouped together and displayed as a frequency plot 1302. Additionally, moving the mouse over an area in the Factor Aggregate View will show the frequency in that sub group at the specific mouse location along the chromosome.

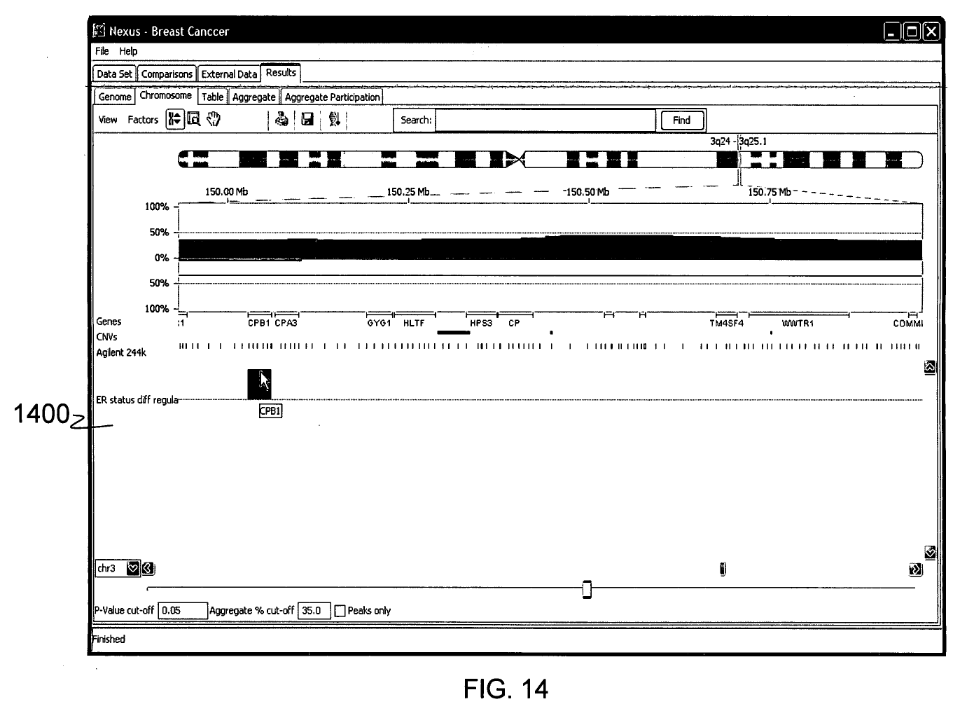

In addition to the comparative genomic hybridization (CGH) data, the user can import data from other genomic or proteomic sources. For example, the user can specify genes differentially regulated in different conditions. As shown in FIG. 14, the user interface allows the user to change the samples view area 1400 to show the differentially regulated genes. The differentially regulated genes can be illustrated using any suitable technique. As a non-limiting example, the display will show up regulation as a bar above the median line and down regulation as a bar below the median line. Different user selected colors can be assigned to each condition, while the extent of the bar is related to gene location. If plotting exon level data, exons can be highlighted as opposed to the whole gene. The same process can be used to visualize methylation, promoter binding location, etc., coming from different sources. Moving the mouse over the segment provides additional information about the measurement. For example, in the case of gene expression, moving the mouse over the segment shows the gene symbol, the p-value, and log ratio values (if available).

For further information related to calculating the copy number, clustering genomic data, analysis of the copy number, and other computational techniques for analysis and use with the present invention, please see attached Appendices A through E, which are papers by the inventors of the present invention. Appendix A is a paper entitled, “Copy Number Computation.” Appendix B is a paper entitled, “Integrated Analysis of Copy Number and Expression Data.” Appendix C is a paper entitled, “Application of Gene Set Enrichment Analysis to DNA Copy Number Data.” Appendix D is a paper entitled, “Clustering Genomic Profiles.” Appendix E is a paper by the inventors of the present invention, entitled, “SNPRank: Segmentation from SNP Data.” Appendices A through E include further details of the present invention and are incorporated by reference as though fully set forth herein.

Additionally, Appendix F, which is incorporated by reference as though fully set forth herein, is a user's manual of a system incorporating the present invention. It should be understand that Appendix F includes descriptions of features and functions of the present invention and is to be used in conjunction with this section to assist the reader in understanding the present invention.

Finally, as can be appreciated by one skilled in the art, the present invention is incorporated into a computer program product that that causes a computer to perform the operations listed above. In other words, the present invention can be embodied as a software program with the features and functionality as described herein. Appendix F includes further descriptions of such a program with corresponding features and functionality.

What is claimed is:

1. A method for analysis and visualization of genomic data, comprising acts of:
selecting at least one individual sample, the sample having chromosomal data representing a genome with a chromosome and including chromosomal measurements of at least one event at a particular location on the chromosome;
generating a frequency of event based on the selected sample, the frequency of event being a frequency of occurrence of the event in the selected sample;
selecting at least one annotation, the annotation including chromosomal region specific information as related to the chromosome; and
displaying the chromosomal data, the annotation, and the frequency of event on a display, thereby allowing a user to view chromosomal region specific information with respect to a particular chromosomal event.

2. A method as set forth in claim 1, wherein the event is a gain or loss of chromosomal copies in the selected sample as compared against a reference chromosomal sample, such that the chromosomal measurements represent chromosomal copies that are gained or lost.

3. A method as set forth in claim 2, further comprising an act of zooming into a selected region of the genome to illustrate chromosomal measurements in the selected region, a corresponding frequency of event in the selected region, and corresponding chromosomal region specific information.

4. A method as set forth in claim 3, wherein the gains and losses of chromosomal copies are displayed as bars having heights that extend from a median line, where the median line represents the reference chromosomal sample and the height of the bars represent copies that are gained or lost from the reference chromosomal sample.

5. A method as set forth in claim 4, further comprising an act of selecting a plurality of samples such that the frequency of event is based on the selected samples, with the frequency of event being a frequency of occurrence of the event across the selected samples.

6. A method as set forth in claim 5, further comprising acts of:
selecting a particular chromosomal event and location from the display of the frequency of event, where the chromosomal event at the selected location spans a region of the chromosome, the spanned region having a span length; and
sorting the samples according to each sample's span length with respect to the selected event.

7. A method as set forth in claim 6, wherein in the act of selecting a plurality of samples, each sample is labeled with at least one factor having a factor value, and further comprising acts of:
selecting a factor with respect to the selected samples;
grouping the selected samples such that the selected samples having the same factor values are grouped together; and
generating and displaying a frequency of event for each group of samples.

8. A method as set forth in claim 1, wherein the event is an chromosomal event selected from a group consisting of an allele gain or loss in the selected sample as compared against a reference chromosomal sample, gene expression and determining if the gene is up regulated or down regulated, a methylated event and determining if the gene is hyper or hypo methylated, and a binding event and determining if there exists a promoter binding or promoter unbinding.

9. A computer program product for analysis and visualization of genomic data, the computer program product comprising computer-readable instruction means stored on a computer-readable medium that are executable by a computer having a processor for causing the processor to perform operations of:
selecting at least one individual sample, the sample having chromosomal data representing a genome with a chromosome and including chromosomal measurements of at least one event at a particular location on the chromosome;
generating a frequency of event based on the selected sample, the frequency of event being a frequency of occurrence of the event in the selected sample;
selecting at least one annotation, the annotation including chromosomal region specific information as related to the chromosome; and
displaying the chromosomal data, the annotation, and the frequency of event on a display, thereby allowing a user to view chromosomal region specific information with respect to a particular chromosomal event.

10. A computer program product as set forth in claim 9, wherein the event is a gain or loss of chromosomal copies in the selected sample as compared against a reference chromosomal sample, such that the chromosomal measurements represent chromosomal copies that are gained or lost.

11. A computer program product as set forth in claim 10, further comprising instruction means for causing the processor to perform an operation of zooming into a selected region of the genome to illustrate chromosomal measurements in the selected region, a corresponding frequency of event in the selected region, and corresponding chromosomal region specific information.

12. A computer program product as set forth in claim 11, wherein the gains and losses of chromosomal copies are displayed as bars having heights that extend from a median line, where the median line represents the reference chromosomal sample and the height of the bars represent copies that are gained or lost from the reference chromosomal sample.

13. A computer program product as set forth in claim 12, further comprising instruction means for causing the processor to perform an operation of selecting a plurality of samples such that the frequency of event is based on the selected samples, with the frequency of event being a frequency of occurrence of the event across the selected samples.

14. A computer program product as set forth in claim 13, further comprising instruction means for causing the processor to perform operations of:
selecting a particular chromosomal event and location from the display of the frequency of event, where the chromosomal event at the selected location spans a region of the chromosome, the spanned region having a span length; and
sorting the samples according to each sample's span length with respect to the selected event.

15. A computer program product as set forth in claim 14, wherein in selecting a plurality of samples, each sample is labeled with at least one factor having a factor value, and further comprising operations of:
selecting a factor with respect to the selected samples;
grouping the selected samples such that the selected samples having the same factor values are grouped together; and
generating and displaying a frequency of event for each group of samples.

16. A computer program product as set forth in claim 9, wherein the event is an chromosomal event selected from a group consisting of an allele gain or loss in the selected sample as compared against a reference chromosomal sample, gene expression and determining if the gene is up regulated or down regulated, a methylated event and determining if the gene is hyper or hypo methylated, and a binding event and determining if there exists a promoter binding or promoter unbinding.

17. A system for analysis and visualization of genomic data, the system comprising on or more processors configured to perform operations of:
selecting at least one individual sample, the sample having chromosomal data representing a genome with a chromosome and including chromosomal measurements of at least one event at a particular location on the chromosome;
generating a frequency of event based on the selected sample, the frequency of event being a frequency of occurrence of the event in the selected sample;
selecting at least one annotation, the annotation including chromosomal region specific information as related to the chromosome; and
displaying the chromosomal data, the annotation, and the frequency of event on a display, thereby allowing a user to view chromosomal region specific information with respect to a particular chromosomal event.

18. A system as set forth in claim 17, wherein the event is a gain or loss of chromosomal copies in the selected sample as compared against a reference chromosomal sample, such that the chromosomal measurements represent chromosomal copies that are gained or lost.

19. A system as set forth in claim 18, wherein the one or more processors are further configured to perform an operation of zooming into a selected region of the genome to illustrate chromosomal measurements in the selected region, a corresponding frequency of event in the selected region, and corresponding chromosomal region specific information.

20. A system as set forth in claim 19, wherein the gains and losses of chromosomal copies are displayed as bars having heights that extend from a median line, where the median line represents the reference chromosomal sample and the height of the bars represent copies that are gained or lost from the reference chromosomal sample.

21. A system as set forth in claim 20, wherein the one or more processors are further configured to perform an operation of selecting a plurality of samples such that the frequency of event is based on the selected samples, with the frequency of event being a frequency of occurrence of the event across the selected samples.

22. A system as set forth in claim 21, wherein the one or more processors are further configured to perform operations of:
selecting a particular chromosomal event and location from the display of the frequency of event, where the chromosomal event at the selected location spans a region of the chromosome, the spanned region having a span length; and
sorting the samples according to each sample's span length with respect to the selected event.

23. A system as set forth in claim 22, wherein selecting a plurality of samples, each sample is labeled with at least one factor having a factor value, and wherein the one or more processors are further configured to perform operations of:
selecting a factor with respect to the selected samples;
grouping the selected samples such that the selected samples having the same factor values are grouped together; and
generating and displaying a frequency of event for each group of samples.

24. A system as set forth in claim 17, wherein the event is an chromosomal event selected from a group consisting of an allele gain or loss in the selected sample as compared against a reference chromosomal sample, gene expression and determining if the gene is up regulated or down regulated, a methylated event and determining if the gene is hyper or hypo methylated, and a binding event and determining if there exists a promoter binding or promoter unbinding.

25. A method for measuring similarity between samples based on genomic data, comprising acts of:
selecting a plurality of individual samples, each sample having chromosomal data representing a genome with a chromosome and including chromosomal measurements of at least one event at a particular location on the chromosome;
generating a frequency of event for each sample, the frequency of event being a frequency of occurrence of the event in the selected sample;
generating an aggregate profile of the genome, the aggregate profile formed of a plurality of samples and representing a percentage of samples having a particular event at each location along the genome;
subdividing the genome into intervals, where each interval has a constant frequency of event;
assigning a weighting function to each interval;
setting a feature vector equal to the weighting function for each sample at each event location;
calculating a distance measure between a pair of samples based on the feature vectors of each sample;
generating a distance matrix showing a distance between any pair of samples; and
clustering the samples based on the distance matrix such that samples with distances below a predetermined threshold are clustered together.

26. A method for integrated analysis of copy number and expression data, comprising acts of:
selecting a genome of interest, the genome of interest having a total of N genes;
selecting a region R with a copy number change greater than a predetermined threshold, the region R having a total of X genes that fall completely within region R or partly cover region R;
identifying Y genes that are to be differentially regulated within region R; and
determining if the Y genes that are to be differentially regulated are differentially regulated at a rate greater than pure chance according to the following:
wherein the probability of drawing X genes at random from the original population and ending up with exactly Y differentially expressed genes is:
$$\frac{( \begin{matrix}M \\ Y\end{matrix} ) ( \begin{matrix}N - M \\ X - Y\end{matrix} )}{( \begin{matrix}N \\ X\end{matrix} )}$$
such that the probability (p-value) of getting at least Y differentially expressed genes is:
$${\sum }_{j = Y}^{X} \frac{( \begin{matrix}M \\ j\end{matrix} ) ( \begin{matrix}N - M \\ X - j\end{matrix} )}{( \begin{matrix}N \\ X\end{matrix} )} ; and$$
calculating a false discover rate corrected Q-value using the p-value.
